(19) United States
(12) Patent Application Publication
(10) Pub. No.: US 2022/0354863 A1
(43) Pub. Date: Nov. 10, 2022

(54) METHODS FOR THE PROPHYLACTIC TREATMENT OF CANCER IN PATIENTS SUFFERING FROM PANCREATITIS

(71) Applicants: INSERM (INSTITUT NATIONAL DE LA SANTÈ ET DE LA RECHERCH MÉDICALE), Paris (FR); UNIVERSITÉ PAUL SABATIER TOULOUSE III, Toulouse (FR)

(72) Inventor: Julie Guillermet-Guibert, Paris (FR)

(21) Appl. No.: 17/624,038

(22) PCT Filed: Jul. 1, 2020
(86) PCT No.: PCT/EP2020/068530

§ 371 Date: Dec. 30, 2021

(30) Foreign Application Priority Data
Jul. 2, 2019 (EP) 19305900.3

(51) Int. Cl. A61K 31/553; A61P 35/00

(52) U.S. Cl. CPC A61K 31/553; A61P 35/00

(57) ABSTRACT

The molecular determinants of chronic pancreatitis leading to pancreatic cancer are underexplored. Genetic or pharmacological inactivation of signaling enzyme PBKa prevents pancreatic fibroinflammatory reaction, while increasing its regenerative capacities, which makes PBKa inhibitors an anti-cancer prevention dmg for these patients. Thus the present invention relates to a method for the prophylactic treatment of cancer in a patient suffering from pancreatitis comprising administering to the patient a therapeutically effective amount of a PBKa- selective inhibitor.

### FIELD OF THE INVENTION

The present invention is in the field of oncology.

### BACKGROUND OF THE INVENTION

Chronic inflammatory conditions are generally thought to set the soil for cancer initiation and to share common molecular induction pathways with neoplasia, explaining the increased prevalence of cancers in patients with chronic inflammation. Indeed, patients with cirrhosis are at high risk of developing hepatocarcinomas. For exocrine pancreatic tissues, the links are less clear however. Acute pancreatitis is one of the most frequent gastrointestinal causes of hospital admission [1]. Chronic pancreatitis (CP) is lower in incidence, but represents a very invalidating condition with various eatiologies such as alcoholic, hereditary, obstructive, autoimmune CP [2]. Pancreatic ductal adenocarcinoma (PDAC) is one of the top five causes of death by cancer and its incidence is on the rise, particularly in <65y population. The burden of all these pancreatic disorders is expected to increase over time. They also share common events of transformation of the parenchyma, in particular the transdifferentiation of acinar exocrine cells into duct cells defining acinar-to-ductal metaplasia (ADM). Nonetheless, patients with hereditary pancreatitis have high risk of developing cancer, and the risk associated between chronic pancreatitis from all aetiologies and pancreatic cancer represents a serious complication for these patients[1, 3] [2] [4]. There may also be other inflammatory conditions that predispose to pancreatic tumor development, such as diabetes. Both type 1 and type 2 diabetes are conditions that have been intimately linked with inflammation (either as the cause or the consequence of destruction or impairment of insulin secreting cells) [5] and with changes in pancreatic cell differentiation [6]. Evidence has been accumulating for an increased risk of pancreatic cancer in subjects with longstanding (>5 years) diabetes [7]. It was even suggested that insulin-producing cells under inflammatory conditions can generate PDAC [8]. Understanding early molecular events occurring during pancreatic inflammatory conditions is critical to help the treatment of patients at higher risk of developing pancreatic cancer.

Phosphoinositide-3-kinases (PI3Ks) act by producing signaling lipids at the plasma membrane. These lipids are known to activate downstream effectors such as Akt [9] by a cascade of phosphorylation events, leading to the regulation of protein synthesis, cell proliferation, cell survival, cell growth, cell migration, cell metabolism, actin remodelling, but also gene expression. Inhibition of all PI3Ks by Wortmaninn or of the immune-restricted PI3Kγ prevents the induction of acute pancreatitis [10] by reducing immune cell recruitment. Mutated Kras, the most frequent initiating mutation occurring in pancreatic cancer, is found in 90% of pancreatic cancer patients and the research of KRAS mutations within endoscopic ultrasound-fine needle aspiration/biopsies (EUS-FNAB) improves the diagnosis of PDAC in pancreatitis context [11]. PI3Kα is critical for the induction of pancreatic cancer by oncogenic Kras in the presence of mutated p53 and/or concomitant inflammation [12, 13]. Inflammation was shown to prevent oncogene-induced senescence and thus to be permissive for pancreatic cancer initiation [14].

### SUMMARY OF THE INVENTION

As defined by the claims, the method of the present invention relates to methods for the prophylactic treatment of cancer in patients suffering from pancreatitis.

### DETAILED DESCRIPTION OF THE INVENTION

The molecular determinants of chronic pancreatitis leading to pancreatic cancer are underexplored. Genetic or pharmacological inactivation of signaling enzyme PI3Kα prevents pancreatic fibroinflammatory reaction, while increasing its regenerative capacities, which makes PI3Kα inhibitors an anti-cancer prevention drug for these patients.

Thus the present invention relates to a method for the prophylactic treatment of cancer in a patient suffering from pancreatitis comprising administering to the patient a therapeutically effective amount of a PI3Kα-selective inhibitor.

As used herein, the term “pancreatitis” has its general meaning in the art and refers to a variety of diseases in which the pancreas becomes inflamed. Pancreatitis is thus inflammation of the pancreas that progresses from acute (sudden onset; duration <6 months) to recurrent acute (>1 episode of acute pancreatitis) to chronic (duration >6 months). Chronic pancreatitis (CP) occurs most commonly after one or more episodes of acute pancreatitis and involves ongoing or recurrent inflammation of the pancreas, often leading to extensive scarring or fibrosis. CP causes progressive and irreversible damage to the pancreas and surrounding tissues. Calcification of pancreatic tissues is common and often diagnostic of CP. In over 70% of cases, CP is associated with excessive and prolonged alcohol consumption. While alcoholism is the most common cause of CP, other causes include metabolic disorders and, more rarely, genetic disposition (hereditary pancreatitis).

As used herein the term “pancreatic cancer” or “pancreas cancer” as used herein relates to cancer which is derived from pancreatic cells. In particular, pancreatic cancer included pancreatic adenocarcinoma (e.g., pancreatic ductal adenocarcinoma) as well as other tumors of the exocrine pancreas (e.g., serous cystadenomas), acinar cell cancers, and intraductal papillary mucinous neoplasms (IPMN).

The terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

As used herein, the term “PI3K” has its general meaning in the art and refers to a phosphoinositide 3-kinase. PI3Ks belong to a large family of lipid signaling kinases that phosphorylate phosphoinositides at the D3 position of the inositol ring (Cantley, Science, 2002, 296(5573):1655-7). PI3Ks are divided into three classes (class I, II, and III) according to their structure, regulation and substrate specificity. Class I PI3Ks, which include PI3Kα, PI3Kβ, PI3Kγ, and PI3Kδ, are a family of dual specificity lipid and protein kinases that catalyze the phosphorylation of phosphatidylinosito-4,5-bisphosphate (PIP2) giving rise to phosphatidylinosito-3,4,5-trisphosphate (PIP3). PIP3 functions as a second messenger that controls a number of cellular processes, including growth, survival, adhesion and migration. All four class I PI3K isoforms exist as heterodimers composed of a catalytic subunit (p110) and a tightly associated regulatory subunit that controls their expression, activation, and subcellular localization. PI3Kα, PI3Kβ, and PI3Kδ associate with a regulatory subunit known as p85 and are activated by growth factors and cytokines through a tyrosine kinase-dependent mechanism (Jimenez, et al., J Biol Chem., 2002, 277(44):41556-62) whereas PI3Kγ associates with two regulatory subunits (p101 and p84) and its activation is driven by the activation of G-protein-coupled receptors (Brock, et al., J Cell Biol., 2003, 160(1):89-99).

Non-limiting examples of s are disclosed in Schmidt-Kittler et al., Oncotarget (2010) 1(5):339-348; Wu et al., Med. Chem. Comm. (2012) 3:659-662; Hayakawa et al., Bioorg. Med. Chem. (2007) 15(17): 5837-5844; and PCT Patent Application Nos. WO2013/049581 and WO2012/052745, the contents of which are herein incorporated by reference in their entireties. In particular non-limiting embodiments, the PI3Kα-selective inhibitor is derived from imidazopyridine or 2-aminothiazole compounds. Further non-limiting examples include those described in William A Denny (2013) Phosphoinositide 3-kinase α inhibitors: a patent review, Expert Opinion on Therapeutic Patents, 23:7, 789-799. Further non-limiting examples include BYL719, INK-1114, INK-1117, NVP-BYL719 (Alpelisib), SRX2523, LY294002, PIK-75, PKI-587, A66, CH5132799 and GDC-0032 (taselisib). One inhibitor suitable for the present invention is the compound 5-(2,6-di-morpholin-4-yl-pyrimidin-4-yl)-4-trifluoromethyl-pyridin-2-ylamine that is described in WO2007/084786, which is hereby incorporated by reference in its entirety hereto. Another inhibitor suitable for the present invention is the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) that is described in WO 2010/029082, which is hereby incorporated by reference in its entirety hereto.

In some embodiments, the PI3Kα-selective inhibitor is an inhibitor ofPI3Kα expression. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of PI3KA mRNA by binding thereto and thus preventing protein translation or increasing mRNA degradation, thus decreasing the level of PI3KA, and thus activity, in a cell. For example, antisense oligonucleotides of at least about 15 bases and complementary to unique regions of the mRNA transcript sequence encoding PI3KA can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. PI3KA gene expression can be reduced by contacting a patient or cell with a small double stranded RNA (dsRNA), or a vector or construct causing the production of a small double stranded RNA, such that PI3KA gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing PI3KA. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. In a particular embodiment, the endonuclease is CRISPR-cas. In some embodiment, the endonuclease is CRISPR-cas9, which is from Streptococcus pyogenes. The CRISPR/Cas9 system has been described in U.S. Pat. No. 8,697,359 B1 and US 2014/0068797. In some embodiment, the endonuclease is CRISPR-Cpf1, which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the patient's size, the severity of the patient's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the PI3Kα-selective inhibitor is administered to the patient in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene- block polymers, polyethylene glycol and wool fat. For use in administration to a subject, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation.

Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following examples. However, these examples should not be interpreted in any way as limiting the scope of the present invention.

### EXAMPLE

### Material & Methods

### Reagents

Reagents were purchased as follows: A66 from Axon Medchem (in vitro IC50 in nM: p110α: 32; β: >12500; δ: >1250; γ: 3480; mTOR: >5000) [41]; Caerulein, Dexamethasone (D1756) from Sigma; EGF from R&D Systems (236-EG-200). GDC0326 also called PI3Kiα was a gift from Genentech (in vitro IC50 in nM: p110α: 0.2; β: 133; δ: 20; γ: 51; C2β: 261; C2α>10 μM; vps34: 2840; Kᵢ mTOR in nM: 4300)[42].

### Human Pancreatic Samples

Human pancreatitis sample were collected according French and European legislation. Pancreatic samples were retrospectively retrieved from the Pathology Department of Beaujon Hospital, Clichy. One paraffin-embedded block of pancreatitis was selected in each case, corresponding to obstructive CP associated with benign lesions or adenocarcinoma (n=10), alcoholic CP (n=4), auto-immune CP (n=1) or CP with unknown origin (n=2). Five 4 μm sections were performed on each block, for hematoxylin-eosin. Frozen material from pancreatitis patients and pancreatic adenocarcinoma patients containing >40% epithelial cells (CRB, Toulouse, IUCT-O) was lysed for WB.

### Mice

The LSL-Kras^{G12D} (from D Tuveson, Mouse Models of Human Cancers Consortium repository (NCI-Frederick, USA), Pdx1-cre (from DA Melton, Harvard University, Cambridge, Mass., USA) [43], p110α^{lox} (from B. Vanhaesebroeck, UCL, London), strains were interbred on mixed background (CD1/C57B16) to obtain compound mutant Pdx1-Cre;LSL-Kras^{G12D} (named KC), pdx1-Cre;p110α^{lox/lox} (Pdx1-Cre;p110α^{lox/lox} were named αⁱⁿC; pdx1-Cre;LSL-Kras^{G12D};p110α^{lox/lox} were named KαⁱⁿC). Littermates not expressing Cre as well as Pdx1-Cre and p110α^{lox/lox} mice of the same age were used as control. All procedures and animal housing are conformed to the regulatory standards and were approved by the Ethical committee according to European legislation translated to French Law as Décret 2013-118 1 Feb. 2013 (APAFIS 3601-2015121622062840). Genotyping was performed as described: the genetically modified allele is called p110α^{lox} or p110α^{ΔDFG} (here named Rec) after Cre recombination. DFG is a conserved motif in the activation loop of the p110α kinase domain critical for its catalytic activity. The gene targeting strategy used is different from a traditional conditional knock-out strategy. Instead, it is deleting two exons in the catalytic domain of Pik3ca in the 3′ part of the gene, allowing expression of a truncated inactive p110α after recombination. Primers corresponds to post-cre primers used to verify the presence of recombined allele ΔDFG: ma9: -ACACACTGCATCAATGGC; ma5: GCTGCCGAATTGCTAGGTAAGC; annealing temperature: 65° C.; recombined ΔDFG-544 bp, wild type (+) or unrecombined lox- >10 kbp amplicon. Genotyping of tail or pancreas samples was performed after DNA extraction with Sigma kit (XNAT-100RXN).

### Caerulein-Induced Pancreatic Injury

Pancreatic injury was induced on young mice (8-12 weeks) by a series of six hourly intra-peritoneal injections of caerulein (75 μg/kg of body weight) that was repeated after 48 h in the presence or absence of GDC0326 (10 mg/kg), for 3 weeks. Animals were euthanized 1, 3, 7 or 17 days later, and recombination verified before analysis as described in [44, 45]. GDC0326 was resuspended in MCT (0.5% (w/v) methylcellulose and 0.2% (w/v) Tween-80 solution (Sigma) and administrated by gavage every morning (concentrations were calculated so as not to reach the maximal volume of 200 μL). Amylase measurement was performed using Phadebas kit in plasma samples.

### Histology and Immunostaining

Immunostainings were conducted using standard methods on formalin-fixed, paraffin-embedded tissues, including both mice and human pancreas.

### In Vitro Culture of Acinar Cells and Treatments

AR42J-B13 cells (a kind gift from Timo Otonkoski, University of Helsinki, Finland), a rat pancreatic acinar cell line, were cultured in Dulbecco's Modified Eagle's Medium with 1 g of glucose (Sigma—D6046) containing 10% FBS (Sigma). Acinar-to-ductal transdifferentiation was induced with 1 μM Dexamethasone and 20 ng/ml EGF (for 5 days with or without the a-selective inhibitor A66 (5 μM) [46]. The culture medium containing diluted agents and inhibitors was changed every 48 h.

### RT-qPCR Analyses

Cells were harvested on ice, washed twice with cold PBS, collected, and frozen at −80° C. RNA of cell pellets was isolated according TRIzol protocol (Life Technologies). RT-qPCR reactions were carried out using RevertAid H Minus Reverse Transcriptase (Thermo Fisher) and SsoFast EvaGreen Supermix (Bio-Rad) according to the manufacturers' instructions. The list of primers is the following.

| | | |
|---|---|---|
| CK19 | 5′-TCCACACTAC | 5′-CTTCCAGGGCAGCT |
| | GCAGATCCAG-3′ | TTCATGC-3 |
| | (SEQ ID NO : 1) | (SEQ ID NO : 2)′ |

### Western Blot Analysis

Cells were harvested on ice, washed twice with cold PBS, collected, and frozen at −80° C. Dry pellets of cells were lysed in 50 mM Tris-HCl, pH 7.5, 150 mM NaCl, 1 mM EDTA, 1% Triton-X100 (SIGMA) supplemented with protease and phosphatase inhibitors (sodium orthovanadate (SIGMA), 1 mM DTT, 2 mM NaF (SIGMA) and cOMPLETE Mini Protease Inhibitor Cocktail (ROCHE). Protein concentration was measured using BCA Protein Assay kit (Interchim), and equal amounts of proteins were subjected to SDS-PAGE and transferred onto nitrocellulose membrane (BioTraceNT; Pall Corp). Membranes were washed in Tris Buffered Saline supplemented with 0.1% Tween-20 (TBS-T) then saturated in TBS-T with 5% non-fat dry milk, incubated overnight with primary antibodies in TBS-T with 5% BSA, washed and revealed according to Cell Signaling Technology protocol. Western blotting was conducted using standard methods with antibodies as described in the Supplemental Table 1.

### Transcriptomics and Bioinformatic Analysis

PI3Kα gene signature was designed as the intersection of genes up- and down-regulated by shRNA against PIK3CA in a human PIK3CA mutated breast cancer cell line [47] and PI3ka_human_LINCS_CMAP and PI3Ka_human_mTOR_CMAP LINCS gene signatures [48]. Amongst the publicly available micro-array repositories, we selected transcriptional profiling datasets of normal pancreas, chronic pancreatitis and pancreatic cancer tissues, including 8 normal, 9 pancreatitis (alcoholic or autoimmune), 5 stroma of chronic pancreatitis (undocumented aetiology) and 7 adenocarcinoma. Published data on human samples were retrieved from public databases (E6MEXP-804[49], E-MEXP-1121[50], E-TABM-145[51]) from compatible platforms, normalized using RMA method (R 3.2.3, bioconductor version 3.2), collapsed (collapse microarray), filtered (SD>0.25), and statistically tested using an ANOVA test corrected with Benjamini & Hochberg method (BH). Published murine mRNA expression during experimental pancreatitis (GSE65146) was analyzed by TTCA package of R (kindly corrected by its conceptor) and normalized with SCAN [52]. Murine PI3Kα targets was validated by [53] using shRNA against PIK3CA in murine KP cell line (mutated Kras, mutated p53 C57/B6 syngenic lung cancer cell line); murine inflammatory signature was designed by Kong et al [27]. Putative PI3Kα targets were identified by STRING (Search Tool for the Retrieval of Interacting Genes/Proteins, string-db.org) with experimental and text mining data.

### Statistics

Experimental data provided at least three biological replicates. Statistical analyses were performed with GraphPad Prism using the T-tests (paired test): *P<0.05, **P<0.01, ***P<0.001. Non-significant (ns) if P>0.05.

### Results

### Pharmacological Inactivation of PI3Kα Prevents the Maintenance of Caerulein-Induced Preneoplastic Lesions and Intralobular Fibrosis in an Oncogenic Kras Context

KrasG12D mutation is the most frequent driving mutation, present in more than 80% of all pancreatic ductal adenocarcinomas, [11, 15] and is found in circulating DNA in chronic pancreatitis patients developping cancer [16]. Given the role of PI3Kα in initiating cancer and acinar-to-ductal preneoplastic ADM lesions, we hypothetized that PI3Kα activity may allow the maintenance of ADM lesions in presence of oncogenic Kras mutations. We thus subjected WT and KrasG12D mice to two rounds of caerulein to accelerate the induction of cancerogenesis [14]. In this oncogenic Kras context, lesions comprised of PanIN or ADM surrounded by fibrotic area triggered by caerulein were maintained during the course of the experiment and are quantified and shown at day 17. As shown previously, activation of the downstream targets of Akt was restricted to the epithelial lesions. Strikingly, treatment with a selective PI3Kα-targeting drug (GDC-0326) during the phase of consolidation of preneoplastic lesions in KrasG12D background led to an absence of p-Akt Subsrate staining and a significant decrease of lesion area compared to caerulein-treated KC mice that received vehicle. In addition, PI3Kα inactivation also reversed the activation of the stromal compartment as assessed by immune cell staining CD18, collagen deposit staining by picrosirius red.

Overall, our data demonstrate that PI3Kα signaling sustains the maintenance of pre-neoplastic lesions and of the intralocular fibrosis induced by oncogenic mutation in an inflammatory context, possibly via both via a cell autonomous manner and a non-cell autonomous manner.

### PI3Kα Genetic Inactivation in Epithelial Lineage without Oncogenic Kras is Sufficient to Delay the Onset of Pancreatic Lesions in a Model of Chronic Pancreatitis

To deconvolute the importance of PI3Ka activity in pancreatic stromal modifications, we used a model of chronic pancreatitis, where exocrine cell dysfunction lead to a progressive replacement of exocrine parenchyma by a fibro-inflammatory reaction. We confirmed that PI3K activity was increased in both chronic pancreatitis patients and pancreatic cancer patients, and that PI3Kα were expressed at similar levels in all samples. We subjected mice to eight series (Chronic pancreatitis) of caerulein injections[17]. Caerulein is a stable analog of cholecystokinin (CCK) that when injected intraperitoneally at supramaximal doses in mice provokes acinar cell hypersecretion of digestive enzymes and a leakage of the pancreatic epithelial barrier, as assessed through measurement of plasmatic amylase. To investigate the role of PI3Kα in pancreatic epithelial cells, we inactivated this enzyme with a genetic approach designed to prevent compensations between isoforms of PI3Ks. Cre recombinase expression in Pdx1-positive cells induces the deletion of two exons encoding the catalytic domain of PI3Kα, leading to a recombined allele of pik3ca . While pancreatic cell-specific genetic inactivation of PI3Kα did not prevent hypersecretion of acinar cells as measured by similar levels of intraplasmatic amylase at early times points after caerulein injections, PI3Kα inactivation delayed the onset of pancreatic injury, as assessed by a significantly decreased lesion score, combined with a decreased of pAktsubstrate staining.

These data demonstrate that epithelial PI3Kα activity is sufficient to promote the induction of lesions in the pancreatic parenchyma observed in a repeated stress model and suggests that PI3Kα signaling in pancreatic epithelium extends beyond epithelial lesions to control the global pathological parenchymal modifications.

### Epithelial PI3Kα Positively Controls ADM in a Repetitive Stress Model while Its Inactivation Increases the Number of Proliferating Acinar Cells During Pancreatitis

We first confirmed that acinar cell transdifferentiation induced by repetitive stress could be prevented by PI3Kα inactivation. The number of acinar-to-ductal structures was decreased by PI3Kα genetic inactivation. Similarly, acinar cell atrophy was blocked and loss of amylase expression was prevented in cells that had an acinar morphology in tissues lacking PI3Kα activity. On the other hand, the duct-specific marker cytokeratin 19 (CK19) was expressed in an increased number of duct-like structures but presented a moderate basolateral expression in cells with acinar morphology when PI3Kα was inactivated. In line with these data, complete pharmacological inactivation of PI3Kα, confirmed by the absence of P-Akt, only partially prevented the increase of CK19 mRNA levels in an in vitro ADM model. ADM occurs upon re-expression of key pancreatic progenitor factors [18]. Nuclear Sox9 re-expression was significantly decreased by genetic PI3Kα inactivation. Inflammation also lead to an increase of acinar cell proliferation which contributes to pancreatic parenchyma regeneration. Indeed, the repititive stress led to an induction of acinar cell proliferation, as assessed by the increased expression of the proliferation marker Ki67 and concomitant decrease of nuclear CDK inhibitor p27 levels. Interestingly, in this context, we observed that Ki67 index and p27 nuclear expression were significantly increased and decreased, respectively, by PI3Kα inactivation. These data suggest that PI3Kα inhibition largely maintains a regenerative response in the pancreatic exocrine parenchyma, which keeps their acinar morphology and possibly contributing to the prevention of ADM completion.

We finally confirmed that an activation of PI3K pathway in human chronic pancreatitis was specifically observed only in ADM structures.

As we previously found in other context (REF), PI3Ka inactivation changes acinar cell fate and prevents the completion of ADM, measured both morphologically and by the expression of the ductal cell marker CK19, progenitor marker Sox9 and Ki67 proliferative marker.

### Epithelial PI3Kα Positively Controls the Induction of Intralobular Fibrosis

Fibroinflammatory reaction is a common phenotype of chronic pancreatic and pancreatic cancer initiation [19]. To question whether PI3Kα activity in the epithelium controls the activation of the fibroinflammatory reaction observed in pancreatic parenchyma, we next analyzed markers of fibrosis and vascular activation. We observed that inactivation of PI3Kα in the pancreatic epithelium delayed the accumulation of αSMA-positive stromal cells and of extracellular matrix as assessed by Masson's Trichrome staining. Inflammation-induced increased numbers of vascular and lymphatic vessels were unchanged. The recruitment of immune cells as assessed by CD45-positive cells within the pancreatic parenchyma was decreased.

Taken together, these results indicate that PI3Kα activated in ADM contributes to the induction of a systemic fibroinflammatory reaction in chronic pancreatitis.

Epithelial PI3Kα positively controls the proinflammatory signal and prevents serine biosynthesis in acinar cells, leading to the acceleration of the fibro-inflammatory reaction.

To search by which mechanism, epithelial PI3Kα could impact the induction of the firboinflammatory reaction, we next assessed the level of activation of known pathways involved in the initiation of pancreatic inflammation. Interestingly, apoptotic acinar cell death (assessed by cleaved caspase 3) was increased upon PI3Kα inactivation. The expression and nuclear localization of NF-κB in ADM is involved in the pathophysiology of chronic pancreatitis [20, 21]. PI3Kα inactivation prevented nuclear translocation of the NF-κB subunit p65 in ADM, therefore possibly preventing an inflammatory response.

### PI3K/Akt Pathway and PI3Kα Downstream Effectors are Differentially Activated in Chronic Pancreatitis Compared to Pancreatic Adenocarcinoma

PI3Kα has an increased impact on PHGDH expression in chronic pancreatitis as compared to oncogenic Kras-induced pancreatic cancer context, we then validated that the expression of PI3Kα transcriptional targets in both physiopathological conexts, pancreatic cancer and pancreatitis samples could be different. Indeed, differential levels of activation of PI3Kα could activate different downstream effectors depending on the cell type and physiological context leading to a differential gene expression profile. We searched for a differential enrichment in HALLMARK and Reactome signatures. First, we confirmed that in human samples, HALLMARK_PI3K_AKT_MTOR_SIGNALING, HALLMARK_MYC_TARGETS_V2, HALLMARK_G2M_CHECKPOINT and HALLMARK_MYC_TARGETS_V1 belong to the 22 hallmarks significantly differentially expressed in CP as compared to PDAC. Besides, when searching for PI3Kα activation in normal, pancreatitis or pancreatic cancer patients, we observed that all pancreatic cancer patient samples shared a common PI3Kα signature (selective high PTTG1, RRM2, CCNE1, KIF2C, TYMS, MELK, CEP55, CCNB1, CDC20), with 7/22 genes from the gene signature being significantly modified, demonstrating increased PI3Kα activity in PDAC. Most chronic pancreatitis samples clustered together (high MYBL2, FGFR4, BCL2, ERBB2, PHGHD, GRB7) while stromal compartment (microdissected samples) from chronic pancreatitis patients presented a PI3Kα-regulated gene profile similar to normal pancreas. These data confirmed that increased PI3K activity is selective to pancreatic epithelial cells in pathological pancreatic samples. A similar number (6/22) of PI3Kα signature genes was found significantly changed in chronic pancreatitis patients compared to normal samples. The expression of 16/22 PI3Ka target mRNAs was significantly different between PDAC and CP patients. Similarly, the expression of PHGDH was regulated differently in time in WT versus Kras-oncogenic model. Hence, Akt increased phosphorylation is associated with the activation of differential PI3Kα downstream transcriptional effectors in both chronic pancreatitis and PDAC. PHGDH levels were increased in IHC, confirming the importance of this target downstream PI3Ka in chronic pancreatitis pathogenesis.

### Discussion

Determining the precise molecular links between chronic inflammatory disease and pancreatic ductal adenocarcinoma is necessary to predict which patients are at risk of developing this lethal disease. Here, we demonstrate that in epithelial cells the signaling enzyme PI3Kα positively contributes to a detrimental fate of acinar cells in repetitively injured pancreata, which sustains the inflammatory parenchymal fibro-reaction.

Both PI3K and MAPK pathways are activated during pancreatic injury. PI3Kα not only controls actin-cytoskeleton remodeling necessary for ADM [12] but we find that it also positively controls the global transcriptional programming of acinar cells. While pancreas-restricted inhibition of MAPK pathway via short hairpins targeting MEK1/2 or their systemic inhibition via a pharmacological inhibitor trametinib (generic name) prevents the formation of ADM and the turnover of pancreatic parenchyma [22], it is interesting to note from our data that PI3Kα does not positively control the proliferation of acinar cells in this context. Hence, the inhibition of PI3Kα signaling could potentially prevent the formation of precursor lesions in the pancreas while maintaining the regenerative capacities of the pancreas. Strobel et al. suggested early on that the absence of expression of the HPAP reporter in exocrine lineage after pancreatic injury was indicative that acinar-to-ductal or acinar-to-centroacinar transdifferenciation could occur, but that this process did not contribute significantly to exocrine regeneration [23]. ADM could also participate in the intrinsic defense mechanism towards pancreatic injury by reducing intra-tissular or systemic leakage of activated digestive enzymes. Our data also argues against that since no increase in plasmatic levels was observed when ADM formation was blocked. The pancreas does not have a high basal rate of cell proliferation compared to other digestive epithelium such as colon or intestine. Similarly, stem/progenitor cells only represent a small proportion of the pancreas [24]. However, pancreatic injury leads to transient re-expression of progenitor factors in all acinar cells [18, 25]. Recent single-cell analysis uncovered that this increase of proliferating acinar cells comes from heterogenous exocrine cell subsets during pancreatic regeneration [24]. PI3Kα genetic and pharmacological inactivation was previously demonstrated by us and others to prevent oncogenic transformation [12, 13] [26]. We demonstrate here that PI3K inactivation induces an increase of the pool of proliferating cells, while Kong et al recently showed that proliferating acinar cells are refractory to oncogenic Kras-transformation [27]. Understanding this balance is critical to prevent cancer formation in an inflammatory context.

Chronic pancreatitis represents a progressive and potentially irreversible damage to the pancreas. One of the most debilitating features of chronic pancreatitis is the progressive installation of the fibroinflammatory response. Interestingly, we demonstrate that a survival signal, such as PI3Kα activation, is responsible for the early induction of this response by preventing apoptosis. Shifting death responses from necrosis to apoptosis may have a therapeutic value for pancreatitis, possibly by reducing perineural inflammation and the intense pain associated with this pathology [28]. PI3Kα was shown to regulate cell survival in pathological context [29] but also in pancreatic cancer cells [30, 31], via p65-containing NF-κB inactivation. Similarly, truncation of p65 induces cell death in the context of pancreatic inflammation [32]. Macrophage-restricted PI3Kγ also sustains pancreatic inflammation, in acute pancreatitis [33], and in pancreatic cancer [34, 35]. Besides their application in cancer, targeting PI3K signal and here selectively inhibiting PI3Kα could be an excellent strategy in chronic inflammatory conditions [36].

Cancer interception is the active way of combating cancer and carcinogenesis at earlier stages. Acinar cells, albeit to a lesser extent than ductal cells [37], are intrinsically refractory to cancerogenesis; expression of oncogenic Kras is required for cell transformation, but is not sufficient to drive cancerogenesis. In line with that idea, in the oncogenic context, pancreatitis-induced inflammation contributes to pancreatic cancer by inhibiting oncogene-induced senescence [14, 17]. Within the lesions found in chronic pancreatitis patients from various aetiologies, we confirm that ADM structures are frequent [38, 39]. Interestingly, we find that activation of PI3K is selectively found in human chronic pancreatic ADM lesions. Given our demonstration of the critical role of a specific PI3K isoform in the maintenance of these lesions, it is tempting to speculate that this active pathway could account for the small proportion of lesions which progress towards cancer. Small molecule inhibitors of all PI3K and selective of PI3Kα, such as GDC0326, are currently in various phases of clinical trials [40]. We hope that, with the rapidly developing techniques allowing detection of circulating premalignant cells and fragmented DNA [16], we will be able to propose strategies to prevent pancreatic cancer in a population at risk. Pharmacological inhibition of pro-cancer pathways such as those driven by PI3Kα should then be tested as a preventive strategy in patients at risk for pancreatic cancer development.

### REFERENCES

Throughout this application, various references describe the state of the art to which this invention pertains. The disclosures of these references are hereby incorporated by reference into the present disclosure.

1. Yadav, D. and A. B. Lowenfels, The epidemiology of pancreatitis and pancreatic cancer. Gastroenterology, 2013. 144(6): p. 1252-61.

2. Kleeff, J., D. C. Whitcomb, T. Shimosegawa, I. Esposito, M. M. Lerch, T. Gress, J. Mayerle, A. M. Drewes, V. Rebours, F. Akisik, J. E. D. Munoz, and J. P. Neoptolemos, Chronic pancreatitis. Nat Rev Dis Primers, 2017. 3: p. 17060.

3. Whitcomb, D. C., L. Frulloni, P. Garg, J. B. Greer, A. Schneider, D. Yadav, and T. Shimosegawa, Chronic pancreatitis: An international draft consensus proposal for a new mechanistic definition. Pancreatology, 2016. 16(2): p. 218-24.

4. Kirkegard, J., F. V. Mortensen, and D. Cronin-Fenton, Chronic Pancreatitis and Pancreatic Cancer Risk: A Systematic Review and Meta-analysis. Am J Gastroenterol, 2017. 112(9): p. 1366-1372.

5. Shoelson, S. E., L. Herrero, and A. Naaz, Obesity, inflammation, and insulin resistance. Gastroenterology, 2007. 132(6): p. 2169-80.

6. Puri, S., A. E. Folias, and M. Hebrok, Plasticity and dedifferentiation within the pancreas: development, homeostasis, and disease. Cell Stem Cell, 2015. 16(1): p. 18-31.

7. Sah, R. P., S. J. Nagpal, D. Mukhopadhyay, and S. T. Chari, New insights into pancreatic cancer-induced paraneoplastic diabetes. Nat Rev Gastroenterol Hepatol, 2013. 10(7): p. 423-33.

8. Gidekel Friedlander, S. Y., G. C. Chu, E. L. Snyder, N. Girnius, G. Dibelius, D. Crowley, E. Vasile, R. A. DePinho, and T. Jacks, Context-dependent transformation of adult pancreatic cells by oncogenic K-Ras. Cancer Cell, 2009. 16(5): p. 379-89.

9. Vanhaesebroeck, B., J. Guillermet-Guibert, M. Graupera, and B. Bilanges, The emerging mechanisms of isoform-specific PI3K signalling. Nat Rev Mol Cell Biol, 2010. 11(5): p. 329-41.

10. Gukovsky, I., J. H. Cheng, K. J. Nam, O. T. Lee, A. Lugea, L. Fischer, J. M. Penninger, S. J. Pandol, and A. S. Gukovskaya, Phosphatidylinositide 3-kinase gamma regulates key pathologic responses to cholecystokinin in pancreatic acinar cells. Gastroenterology, 2004. 126(2): p. 554-66.

11. Bournet, B., M. Gayral, J. Torrisani, J. Selves, P. Cordelier, and L. Buscail, Role of endoscopic ultrasound in the molecular diagnosis of pancreatic cancer. World J Gastroenterol, 2014. 20(31): p. 10758-68.

12. Baer, R., C. Cintas, M. Dufresne, S. Cassant-Sourdy, N. Schonhuber, L. Planque, H. Lulka, B. Couderc, C. Bousquet, B. Garmy-Susini, B. Vanhaesebroeck, S. Pyronnet, D. Saur, and J. Guillermet-Guibert, Pancreatic cell plasticity and cancer initiation induced by oncogenic Kras is completely dependent on wild-type PI 3-kinase p110alpha. Genes Dev, 2014. 28(23): p. 2621-35.

13. Wu, C. Y., E. S. Carpenter, K. K. Takeuchi, C. J. Halbrook, L. V. Peverley, H. Bien, J. C. Hall, K. E. DelGiorno, D. Pal, Y. Song, C. Shi, R. Z. Lin, and H. C. Crawford, PI3K regulation of RAC1 is required for KRAS-induced pancreatic tumorigenesis in mice. Gastroenterology, 2014. 147(6): p. 1405-16 e7.

14. Guerra, C., A. J. Schuhmacher, M. Canamero, P. J. Grippo, L. Verdaguer, L. Perez-Gallego, P. Dubus, E. P. Sandgren, and M. Barbacid, Chronic pancreatitis is essential for induction of pancreatic ductal adenocarcinoma by K-Ras oncogenes in adult mice. Cancer Cell, 2007. 11(3): p. 291-302.

15. Bournet, B., F. Muscari, C. Buscail, E. Assenat, M. Barthet, P. Hammel, J. Selves, R. Guimbaud, P. Cordelier, and L. Buscail, KRAS G12D Mutation Subtype Is A Prognostic Factor for Advanced Pancreatic Adenocarcinoma. Clin Transl Gastroenterol, 2016. 7: p. e157.

16. Yang, S., S. P. Che, P. Kurywchak, J. L. Tavormina, L. B. Gansmo, P. Correa de Sampaio, M. Tachezy, M. Bockhorn, F. Gebauer, A. R. Haltom, S. A. Melo, V. S. LeBleu, and R. Kalluri, Detection of mutant KRAS and TP53 DNA in circulating exosomes from healthy individuals and patients with pancreatic cancer. Cancer Biol Ther, 2017. 18(3): p. 158-165.

17. Carriere, C., A. L. Young, J. R. Gunn, D. S. Longnecker, and M. Korc, Acute pancreatitis accelerates initiation and progression to pancreatic cancer in mice expressing oncogenic Kras in the nestin cell lineage. PLoS One, 2011. 6(11): p. e27725.

18. Kopp, J. L., G. von Figura, E. Mayes, F. F. Liu, C. L. Dubois, J. P. t. Morris, F. C. Pan, H. Akiyama, C. V. Wright, K. Jensen, M. Hebrok, and M. Sander, Identification of Sox9-dependent acinar-to-ductal reprogramming as the principal mechanism for initiation of pancreatic ductal adenocarcinoma. Cancer Cell, 2012. 22(6): p. 737-50.

19. Algul, H., M. Treiber, M. Lesina, and R. M. Schmid, Mechanisms of disease: chronic inflammation and cancer in the pancreas--a potential role for pancreatic stellate cells? Nat Clin Pract Gastroenterol Hepatol, 2007. 4(8): p. 454-62.

20. Aleksic, T., B. Baumann, M. Wagner, G. Adler, T. Wirth, and C. K. Weber, Cellular immune reaction in the pancreas is induced by constitutively active IkappaB kinase-2. Gut, 2007. 56(2): p. 227-36.

21. Sendler, M., A. Dummer, F. U. Weiss, B. Kruger, T. Wartmann, K. Scharffetter-Kochanek, N. van Rooijen, S. R. Malla, A. Aghdassi, W. Halangk, M. M. Lerch, and J. Mayerle, Tumour necrosis factor alpha secretion induces protease activation and acinar cell necrosis in acute experimental pancreatitis in mice. Gut, 2013. 62(3): p. 430-9.

22. Halbrook, C. J., H. J. Wen, J. M. Ruggeri, K. K. Takeuchi, Y. Zhang, M. P. di Magliano, and H. C. Crawford, Mitogen-activated Protein Kinase Kinase Activity Maintains Acinar-to-Ductal Metaplasia and Is Required for Organ Regeneration in Pancreatitis. Cell Mol Gastroenterol Hepatol, 2017. 3(1): p. 99-118.

23. Strobel, O., Y. Dor, J. Alsina, A. Stirman, G. Lauwers, A. Trainor, C. F. Castillo, A. L. Warshaw, and S. P. Thayer, In vivo lineage tracing defines the role of acinar-to-ductal transdifferentiation in inflammatory ductal metaplasia. Gastroenterology, 2007. 133(6): p. 1999-2009.

24. Wollny, D., S. Zhao, I. Everlien, X. Lun, J. Brunken, D. Brune, F. Ziebell, I. Tabansky, W. Weichert, A. Marciniak-Czochra, and A. Martin-Villalba, Single-Cell Analysis Uncovers Clonal Acinar Cell Heterogeneity in the Adult Pancreas. Dev Cell, 2016. 39(3): p. 289-301.

25. Jeannot, P., C. Callot, R. Baer, N. Duquesnes, C. Guerra, J. Guillermet-Guibert, O. Bachs, and A. Besson, Loss of p27Kip1 promotes metaplasia in the pancreas via the regulation of Sox9 expression. Oncotarget, 2015.

26. Baer, R., C. Cintas, N. Therville, and J. Guillermet-Guibert, Implication of PI3K/Akt pathway in pancreatic cancer: When PI3K isoforms matter? Adv Biol Regul, 2015. 59: p. 19-35.

27. Kong, B., P. Bruns, N. A. Behler, L. Chang, A. M. Schlitter, J. Cao, A. Gewies, J. Ruland, S. Fritzsche, N. Valkovskaya, Z. Jian, I. Regel, S. Raulefs, M. Irmler, J. Beckers, H. Friess, M. Erkan, N. S. Mueller, S. Roth, T. Hackert, I. Esposito, F. J. Theis, J. Kleeff, and C. W. Michalski, Dynamic landscape of pancreatic carcinogenesis reveals early molecular networks of malignancy. Gut, 2016.

28. Mareninova, O. A., K. F. Sung, P. Hong, A. Lugea, S. J. Pandol, I. Gukovsky, and A. S. Gukovskaya, Cell death in pancreatitis: caspases protect from necrotizing pancreatitis. J Biol Chem, 2006. 281(6): p. 3370-81.

29. Guillermet-Guibert, J., N. Saint-Laurent, L. Davenne, P. Rochaix, O. Cuvillier, M. D. Culler, L. Pradayrol, L. Buscail, C. Susini, and C. Bousquet, Novel synergistic mechanism for sst2 somatostatin and TNFalpha receptors to induce apoptosis: crosstalk between NF-kappaB and JNK pathways. Cell Death Differ, 2007. 14(2): p. 197-208.

30. Bousquet, C., J. Guillermet-Guibert, N. Saint-Laurent, E. Archer-Lahlou, F. Lopez, M. Fanjul, A. Ferrand, D. Fourmy, C. Pichereaux, B. Monsarrat, L. Pradayrol, J. P. Esteve, and C. Susini, Direct binding of p85 to sst2 somatostatin receptor reveals a novel mechanism for inhibiting PI3K pathway. EMBO J, 2006. 25(17): p. 3943-54.

31. Guillermet, J., N. Saint-Laurent, P. Rochaix, O. Cuvillier, T. Levade, A. V. Schally, L. Pradayrol, L. Buscail, C. Susini, and C. Bousquet, Somatostatin receptor subtype 2 sensitizes human pancreatic cancer cells to death ligand-induced apoptosis. Proc Natl Acad Sci U S A, 2003. 100(1): p. 155-60.

32. Algul, H., M. Treiber, M. Lesina, H. Nakhai, D. Saur, F. Geisler, A. Pfeifer, S. Paxian, and R. M. Schmid, Pancreas-specific RelA/p65 truncation increases susceptibility of acini to inflammation-associated cell death following cerulein pancreatitis. J Clin Invest, 2007. 117(6): p. 1490-501.

33. Fischer, L., A. S. Gukovskaya, J. M. Penninger, O. A. Mareninova, H. Friess, I. Gukovsky, and S. J. Pandol, Phosphatidylinositol 3-kinase facilitates bile acid-induced Ca(2+) responses in pancreatic acinar cells. Am J Physiol Gastrointest Liver Physiol, 2007. 292(3): p. G875-86.

34. Basset, C. and J. Guillermet-Guibert, Attenuating PI3K isoforms in pancreatic cancer: Focus on immune PI3Kgamma. Clin Res Hepatol Gastroenterol, 2017. 41(4): p. 351-353.

35. Kaneda, M. M., P. Cappello, A. V. Nguyen, N. Ralainirina, C. R. Hardamon, P. Foubert, M. C. Schmid, P. Sun, E. Mose, M. Bouvet, A. M. Lowy, M. A. Valasek, R. Sasik, F. Novelli, E. Hirsch, and J. A. Varner, Macrophage PI3Kgamma Drives Pancreatic Ductal Adenocarcinoma Progression. Cancer Discov, 2016. 6(8): p. 870-85.

36. Vanhaesebroeck, B., M. A. Whitehead, and R. Pineiro, Molecules in medicine mini-review: isoforms of PI3K in biology and disease. J Mol Med (Berl), 2016. 94(1): p. 5-11.

37. Bailey, J. M., A. M. Hendley, K. J. Lafaro, M. A. Pruski, N. C. Jones, J. Alsina, M. Younes, A. Maitra, F. McAllister, C. A. Iacobuzio-Donahue, and S. D. Leach, p53 mutations cooperate with oncogenic Kras to promote adenocarcinoma from pancreatic ductal cells. Oncogene, 2016. 35(32): p. 4282-8.

38. Esposito, I., C. Seiler, F. Bergmann, J. Kleeff, H. Friess, and P. Schirmacher, Hypothetical progression model of pancreatic cancer with origin in the centroacinar-acinar compartment. Pancreas, 2007. 35(3): p. 212-7.

39. Rebours, V., P. Levy, J. F. Mosnier, J. Y. Scoazec, M. S. Soubeyrand, J. F. Flejou, B. Turlin, P. Hammel, P. Ruszniewski, P. Bedossa, and A. Couvelard, Pathology analysis reveals that dysplastic pancreatic ductal lesions are frequent in patients with hereditary pancreatitis. Clin Gastroenterol Hepatol, 2010. 8(2): p. 206-12.

40. Pons-Tostivint, E., B. Thibault, and J. Guillermet-Guibert, Targeting PI3K Signaling in Combination Cancer Therapy. Trends Cancer, 2017. 3(6): p. 454-469.

41. Jamieson, S., J. U. Flanagan, S. Kolekar, C. Buchanan, J. D. Kendall, W. J. Lee, G. W. Rewcastle, W. A. Denny, R. Singh, J. Dickson, B. C. Baguley, and P. R. Shepherd, A drug targeting only p110alpha can block phosphoinositide 3-kinase signalling and tumour growth in certain cell types. Biochem J, 2011. 438(1): p. 53-62.

42. Heffron, T. P., R. A. Heald, C. Ndubaku, B. Wei, M. Augistin, S. Do, K. Edgar, C. Eigenbrot, L. Friedman, E. Gancia, P. S. Jackson, G. Jones, A. Kolesnikov, L. B. Lee, J. D. Lesnick, C. Lewis, N. McLean, M. Mortl, J. Nonomiya, J. Pang, S. Price, W. W. Prior, L. Salphati, S. Sideris, S. T. Staben, S. Steinbacher, V. Tsui, J. Wallin, D. Sampath, and A. G. Olivero, The Rational Design of Selective Benzoxazepin Inhibitors of the alpha-Isoform of Phosphoinositide 3-Kinase Culminating in the Identification of (S)-2-((2-(1-Isopropyl-1H-1,2,4-triazol-5-yl)-5,6-dihydrobenzo[f]imidazo [1,2-d][1,4]oxazepin-9-yl)oxy)propanamide (GDC-0326). J Med Chem, 2016. 59(3): p. 985-1002.

43. Gu, G., J. R. Brown, and D. A. Melton, Direct lineage tracing reveals the ontogeny of pancreatic cell fates during mouse embryogenesis. Mech Dev, 2003. 120(1): p. 35-43.

44. Guillermet-Guibert, J., K. Bjorklof, A. Salpekar, C. Gonella, F. Ramadani, A. Bilancio, S. Meek, A. J. Smith, K. Okkenhaug, and B. Vanhaesebroeck, The p110beta isoform of phosphoinositide 3-kinase signals downstream of G protein-coupled receptors and is functionally redundant with p110 gamma. Proc Natl Acad Sci U S A, 2008. 105(24): p. 8292-7.

45. Graupera, M., J. Guillermet-Guibert, L. C. Foukas, L. K. Phng, R. J. Cain, A. Salpekar, W. Pearce, S. Meek, J. Millan, P. R. Cutillas, A. J. Smith, A. J. Ridley, C. Ruhrberg, H. Gerhardt, and B. Vanhaesebroeck, Angiogenesis selectively requires the p110alpha isoform of PI3K to control endothelial cell migration. Nature, 2008. 453(7195): p. 662-6.

46. Al-Adsani, A., Z. D. Burke, D. Eberhard, K. L. Lawrence, C. N. Shen, A. K. Rustgi, H. Sakaue, J. M. Farrant, and D. Tosh, Dexamethasone treatment induces the reprogramming of pancreatic acinar cells to hepatocytes and ductal cells. PLoS One, 2010. 5(10): p. e13650.

47. Bosch, A., Z. Li, A. Bergamaschi, H. Ellis, E. Toska, A. Prat, J. J. Tao, D. E. Spratt, N. T. Viola-Villegas, P. Castel, G. Minuesa, N. Morse, J. Rodon, Y. Ibrahim, J. Cortes, J. Perez-Garcia, P. Galvan, J. Grueso, M. Guzman, J. A. Katzenellenbogen, M. Kharas, J. S. Lewis, M. Dickler, V. Serra, N. Rosen, S. Chandarlapaty, M. Scaltriti, and J. Baselga, PI3K inhibition results in enhanced estrogen receptor function and dependence in hormone receptor-positive breast cancer. Sci Transl Med, 2015. 7(283): p. 283ra51.

48. Zhang, Y., P. Kwok-Shing Ng, M. Kucherlapati, F. Chen, Y. Liu, Y. H. Tsang, G. de Velasco, K. J. Jeong, R. Akbani, A. Hadjipanayis, A. Pantazi, C. A. Bristow, E. Lee, H. S. Mahadeshwar, J. Tang, J. Zhang, L. Yang, S. Seth, S. Lee, X. Ren, X. Song, H. Sun, J. Seidman, L. J. Luquette, R. Xi, L. Chin, A. Protopopov, T. F. Westbrook, C. S. Shelley, T. K. Choueiri, M. Ittmann, C. Van Waes, J. N. Weinstein, H. Liang, E. P. Henske, A. K. Godwin, P. J. Park, R. Kucherlapati, K. L. Scott, G. B. Mills, D. J. Kwiatkowski, and C. J. Creighton, A Pan-Cancer Proteogenomic Atlas of PI3K/AKT/mTOR Pathway Alterations. Cancer Cell, 2017. 31(6): p. 820-832 e3.

49. Lohr, J. M., R. Faissner, D. Koczan, P. Bewerunge, C. Bassi, B. Brors, R. Eils, L. Frulloni, A. Funk, W. Halangk, R. Jesenofsky, L. Kaderali, J. Kleeff, B. Kruger, M. M. Lerch, R. Losel, M. Magnani, M. Neumaier, S. Nittka, M. Sahin-Toth, J. Sanger, S. Serafini, M. Schnolzer, H. J. Thierse, S. Wandschneider, G. Zamboni, and G. Kloppel, Autoantibodies against the exocrine pancreas in autoimmune pancreatitis: gene and protein expression profiling and immunoassays identify pancreatic enzymes as a major target of the inflammatory process. Am J Gastroenterol, 2010. 105(9): p. 2060-71.

50. Ruckert, F., M. Hennig, C. D. Petraki, D. Wehrum, M. Distler, A. Denz, M. Schroder, G. Dawelbait, H. Kalthoff, H. D. Saeger, E. P. Diamandis, C. Pilarsky, and R. Grutzmann, Co-expression of KLK6 and KLK10 as prognostic factors for survival in pancreatic ductal adenocarcinoma. Br J Cancer, 2008. 99(9): p. 1484-92.

51. Vaquerizas, J. M., S. K. Kummerfeld, S. A. Teichmann, and N. M. Luscombe, A census of human transcription factors: function, expression and evolution. Nat Rev Genet, 2009. 10(4): p. 252-63.

52. Albrecht, M., D. Stichel, B. Muller, R. Merkle, C. Sticht, N. Gretz, U. Klingmuller, K. Breuhahn, and F. Matthaus, TTCA: an R package for the identification of differentially expressed genes in time course microarray data. BMC Bioinformatics, 2017. 18(1): p. 33.

53. Yang, Y., Y. H. Ahn, Y. Chen, X. Tan, L. Guo, D. L. Gibbons, C. Ungewiss, D. H. Peng, X. Liu, S. H. Lin, N. Thilaganathan, Wistuba, II, J. Rodriguez-Canales, G. McLendon, C. J. Creighton, and J. M. Kurie, ZEB1 sensitizes lung adenocarcinoma to metastasis suppression by PI3K antagonism. J Clin Invest, 2014. 124(6): p. 2696-708.

1. A method for the prophylactic treatment of cancer in a patient suffering from pancreatitis comprising administering to the patient a therapeutically effective amount of a PI3Kα-selective inhibitor.


SEQUENCE LISTING (2 sequences)

SEQ ID NO: 1 - DNA, 20 bp, Artificial Sequence
  Primer CK19
tccacactacgcagatccag

SEQ ID NO: 2 - DNA, 21 bp, Artificial Sequence
  Primer CK19
cttccagggcagctttcatgc